(19) United States
(12) Patent Application Publication
(10) Pub. No.: US 2019/0072540 A1
(43) Pub. Date: Mar. 7, 2019

(54) ELECTRICAL CHARACTERISTICS MEASUREMENT APPARATUS, ELECTRICAL CHARACTERISTICS MEASUREMENT SYSTEM, ELECTRICAL CHARACTERISTICS MEASUREMENT METHOD, AND PROGRAM

(71) Applicant: Sony Corporation, Tokyo (JP)

(72) Inventors: Marcaurele Brun, Tokyo (JP); Isao Hidaka, Tokyo (JP); Shinji Omori, Chiba (JP)

(73) Assignee: Sony Corporation, Tokyo (JP)

(21) Appl. No.: 16/087,443

(22) PCT Filed: Dec. 22, 2016
(86) PCT No.: PCT/JP2016/088320

§ 371 Date: Sep. 21, 2018

(30) Foreign Application Priority Data
Mar. 28, 2016 (JP) 2016-063461

(51) Int. Cl. G01N 33/49; G01N 27/02; G01N 33/86; G01N 33/487

(52) U.S. Cl. CPC G01N 33/4905; G01N 33/48707; G01N 33/86; G01N 27/026

(57) ABSTRACT

The main purpose of the present technology is to provide a technology capable of reducing the risk of erroneous determination while ensuring real-time properties in the measurement of electrical characteristics of a biological sample.

In this regard, the present technology provides an electrical characteristics measurement apparatus including: at least a measurement unit that measures electrical characteristics of a biological sample over time; an analysis unit that reviews, in real time during the measurement, data related to a temporal change in the electrical characteristics and analyzes a change in state of the biological sample; and a notification unit that issues notification of an analysis result in the analysis unit at a specific time point, in which the analysis unit detects a predetermined feature point from the data related to the temporal change in the electrical characteristics to use data related to the temporal change in a period before and/or after the predetermined feature point.

### TECHNICAL FIELD

The present technology relates to an electrical characteristics measurement apparatus. More specifically, the present technology relates to an electrical characteristics measurement apparatus, an electrical characteristics measurement system, an electrical characteristics measurement method, and a program that are capable of reducing the risk of erroneous determination while ensuring real-time properties in the measurement of electrical characteristics of a biological sample.

### BACKGROUND ART

In the past, the electrical characteristics of biological samples have been measured to determine, from those measurement results, physical properties of the samples or the types of cells or the like included in the samples (e.g., Patent Literature 1). Examples of electrical characteristics to be measured include a complex dielectric constant and its frequency dispersion (dielectric spectrum). In general, the complex dielectric constant and its frequency dispersion are calculated by measuring a complex capacitance and a complex impedance between electrodes while using a solution holder or the like including an electrode for applying a voltage to a solution.

For example, Patent Literature 2 discloses a technology for acquiring information relating to blood clotting from a dielectric constant of the blood, and describes “a blood clotting system analysis apparatus including: a pair of electrodes; application means for applying an alternating voltage to the pair of electrodes at predetermined time intervals; measurement means for measuring a dielectric constant of blood which is positioned between the pair of electrodes; and analysis means for analyzing a degree of the action of a blood clotting system by using the dielectric constant of blood which is measured at the time intervals after the anticoagulant effect acting on the blood is ended.”

### CITATION LIST

### Patent Literature

Patent Literature 1: Japanese Patent Application Laid-open No. 2009-042141

Patent Literature 2: Japanese Patent Application Laid-open No. 2010-181400

### DISCLOSURE OF INVENTION

### Technical Problem

In the analysis of a change in state of a biological sample with conventional testing equipment or the like, for example, in a case where the change in state is blood clotting and where the biological sample is a blood specimen of a patient being operated on, the display of erroneous determination or the display of determination delayed from a suitable timing has a high risk of affecting the treatment of the patient. Therefore, the testing equipment or the like that measures a temporal change in state of the biological sample is strongly requested for real-time properties in analysis, determination, and display in actuality. Specifically, for example, there is a demand to avoid the following risk: a temporal change in signal, which results from an erythrocyte sedimentation rate (sedimentation), is erroneously determined as a signal of blood clotting, and then a blood-clotting start time is calculated and displayed; in a case where the obtained value falls within a normal range, a blood clotting accelerator that is practically necessary for a patient is not prescribed, and the risk of bleeding probably increases.

In this regard, it is a main object of the present technology to provide a technology capable of reducing the risk of erroneous determination while ensuring real-time properties in the measurement of electrical characteristics of a biological sample.

### Solution to Problem

In other words, in the present technology, there is first provided an electrical characteristics measurement apparatus including: at least a measurement unit that measures electrical characteristics of a biological sample over time; an analysis unit that reviews, in real time during the measurement, data related to a temporal change in the electrical characteristics and analyzes a change in state of the biological sample; and a notification unit that issues notification of an analysis result in the analysis unit at a specific time point, in which the analysis unit detects a predetermined feature point from the data related to the temporal change in the electrical characteristics to use data related to the temporal change in a period before and/or after the predetermined feature point.

In the electrical characteristics measurement apparatus according to the present technology, the analysis unit may use pieces of data related to the temporal change. In this case, the analysis unit may detect a time point at which a value at the predetermined feature point exceeds a predetermined threshold value and compare variations in electrical characteristics at the time point between the pieces of data related to the temporal change. Further, the analysis unit may calculate a correlation coefficient between the pieces of data related to the temporal change and analyze whether the correlation coefficient exceeds a predetermined threshold value or not.

In the electrical characteristics measurement apparatus according to the present technology, the analysis unit may analyze whether a value at the predetermined feature point and/or the state of the biological sample at the predetermined feature point are/is matched with a predetermined criterion or not.

In the electrical characteristics measurement apparatus according to the present technology, the biological sample may be a blood sample.

In the electrical characteristics measurement apparatus according to the present technology, the electrical characteristics may be a dielectric constant at a specific frequency.

Further, in the present technology, there is also provided an electrical characteristics measurement system including: at least a measurement unit that measures electrical characteristics of a biological sample over time; an analysis unit that reviews, in real time during the measurement, data related to a temporal change in the electrical characteristics and analyzes a change in state of the biological sample; and a notification unit that issues notification of an analysis result in the analysis unit at a specific time point, in which the analysis unit detects a predetermined feature point from the data related to the temporal change in the electrical characteristics to use data related to the temporal change in a period before and/or after the predetermined feature point.

The electrical characteristics measurement system according to the present technology may further include a server including at least a storage unit that stores the data related to the temporal change in the measurement unit and/or the analysis result in the analysis unit, the server being connected to the measurement unit and/or the analysis unit via a network.

Further, in the present technology, there is also provided an electrical characteristics measurement method including: at least a measuring step of measuring electrical characteristics of a biological sample over time; an analyzing step of reviewing, in real time during the measurement, data related to a temporal change in the electrical characteristics and analyzing a change in state of the biological sample; and a notifying step of issuing notification of an analysis result in the analysis unit at a specific time point, in which the analyzing step including detecting a predetermined feature point from the data related to the temporal change in the electrical characteristics to use data related to the temporal change in a period before and/or after the predetermined feature point.

In the electrical characteristics measurement method according to the present technology, the analyzing step may include using pieces of data related to the temporal change. In this case, the analyzing step may include detecting a time point at which a value at the predetermined feature point exceeds a predetermined threshold value and comparing variations in electrical characteristics at the time point between the pieces of data related to the temporal change. Further, the analyzing step may include calculating a correlation coefficient between the pieces of data related to the temporal change and analyzing whether the correlation coefficient exceeds a predetermined threshold value or not.

In the electrical characteristics measurement method according to the present technology, the analyzing step may also include analyzing whether a value at the predetermined feature point and/or the state of the biological sample at the predetermined feature point are/is matched with a predetermined criterion or not.

In addition, in the present technology, there is also provided a program that causes a computer to function as: a measurement unit that measures electrical characteristics of a biological sample over time; an analysis unit that reviews, in real time during the measurement, data related to a temporal change in the electrical characteristics and analyzes a change in state of the biological sample; and a notification unit that issues notification of an analysis result in the analysis unit at a specific time point, in which the analysis unit detects a predetermined feature point from the data related to the temporal change in the electrical characteristics to use data related to the temporal change in a period before and/or after the predetermined feature point.

### Advantageous Effects of Invention

According to the present technology, it is possible to reduce the risk of erroneous determination while ensuring real-time properties in the measurement of electrical characteristics of a biological sample. Note that the effects described herein are not necessarily limited and may be any of the effects described in the present disclosure.

### BRIEF DESCRIPTION OF DRAWINGS

FIG. 1 is a schematic conceptual diagram schematically showing a concept of an electrical characteristics measurement apparatus 100 according to the present technology.

FIG. 2 is a schematic cross-sectional view schematically showing one form of a biological sample holding unit 110.

FIG. 3 is a drawing substitute graph showing data related to a temporal change at the dielectric constants of 1 MHz and 10 MHz in the case of a typical blood clotting process.

FIG. 4 is a drawing substitute graph showing a first-order derivative of 1 MHz and a first-order derivative of 10 MHz in the case of a typical blood clotting process.

FIG. 5 is a drawing substitute graph showing data related to the temporal change and a difference in first-order derivative in the case of a typical blood clotting process.

FIG. 6 is a drawing substitute graph showing data related to the temporal change at the dielectric constants of 1 MHz and 10 MHz in the case where an erythrocyte sedimentation rate (sedimentation) is abnormal.

FIG. 7 is a drawing substitute graph showing a first-order derivative of 1 MHz and a first-order derivative of 10 MHz in the case where the erythrocyte sedimentation rate (sedimentation) is abnormal.

FIG. 8 is a drawing substitute graph showing data related to the temporal change and a difference in first-order derivative in the case where the erythrocyte sedimentation rate (sedimentation) is abnormal.

FIG. 9 is a drawing substitute graph showing calculation results in the case where tbuffer=12 in the data related to the temporal change shown in FIGS. 3 and 6.

FIG. 10 is a drawing substitute graph showing data related to the temporal change of a blood sample in which the sedimentation of red blood cells progresses very slowly and the blood is not basically clotted.

FIG. 11 is a drawing substitute graph showing analysis results of a first-order derivative at two minutes after T10_1 and a second-order derivative at two minutes after T10_1.

FIG. 12 is a drawing substitute graph showing data related to the temporal change in the case where determination cannot yet be made at the time point of the determination of T10_1 because the blood clotting is very slow.

FIG. 13 is a schematic conceptual diagram schematically showing a concept of an electrical characteristics measurement system 200 according to the present technology.

FIG. 14 is a flowchart showing an example of an electrical characteristics measurement method according to the present technology.

FIG. 15 is a flowchart showing an example of the electrical characteristics measurement method according to the present technology, which is different from that of FIG. 14.

FIG. 16 is a flowchart showing an example of the electrical characteristics measurement method according to the present technology, which is different from those of FIGS. 14 and 15.

FIG. 17 is a flowchart showing an example of the electrical characteristics measurement method according to the present technology, which is different from those of FIGS. 14 to 16.

### MODE(S) FOR CARRYING OUT THE INVENTION

Hereinafter, favorable embodiments for carrying out the present technology will be described with reference to the drawings. Note that the embodiments to be described below illustrate only examples of typical embodiments of the present technology, and the scope of the present technology is not narrowly interpreted by the embodiments. Note that description will be made in the following order.

1. Electrical characteristics Measurement Apparatus 100

### (1) Measurement Unit 1

(a) Biological Sample Holding Unit 110
   (a-1) Container 111
   (a-2) Container Holding Unit 112
(b) Application Unit 120
   (b-1) Electrodes 121a, 121b
   (b-2) Connection Unit 122

### (2) Analysis Unit 2

### [Analysis Example 1 Performed by Analysis Unit 2]

### [Analysis Example 2 Performed by Analysis Unit 2]

### [Analysis Example 3 Performed by Analysis Unit 2]

### (3) Notification Unit 3

### (4) Display Unit 4

### (5) Storage Unit 5

### (6) Measurement Condition Control Unit 6

### (7) Temperature Control Unit 7

### (8) Biological Sample Supply Unit 8

### (9) Agent Supply Unit 9

### (10) Precision Management Unit 10

### (11) Drive Mechanism 11

### (12) Sample Standby Unit 12

### (13) Stirring Mechanism 13

### (14) Others

2. Electrical characteristics Measurement System 200

### (1) Display Unit 201

### (2) User Interface 202

### (3) Server 203

3. Electrical characteristics Measurement method

### [Measurement Method Example 1]

### [Measurement Method Example 2]

### [Measurement Method Example 3]

### [Measurement Method Example 4]

1. Electrical Characteristics Measurement Apparatus 100

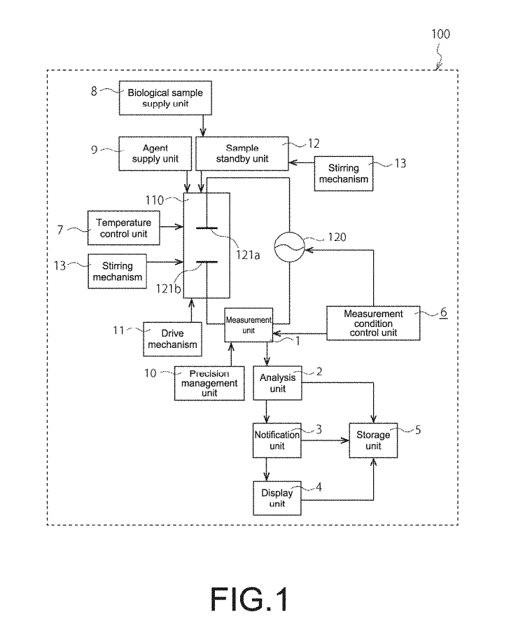

FIG. 1 is a schematic conceptual diagram schematically showing a concept of an electrical characteristics measurement apparatus 100 according to the present technology. The electrical characteristics measurement apparatus 100 according to the present technology roughly includes at least a measurement unit 1, an analysis unit 2, and a notification unit 3. Further, as necessary, the electrical characteristics measurement apparatus 100 may include a display unit 4, a storage unit 5, a measurement condition control unit 6, a temperature control unit 7, a biological sample supply unit 8, an agent supply unit 9, a precision management unit 10, a drive mechanism 11, a sample standby unit 12, a stirring mechanism 13, and the like. Hereinafter, the respective units will be described in detail.

(1) Measurement Unit 1

The measurement unit 1 is a section that measures electrical characteristics of a biological sample S over time. In the present technology, the biological sample S is not particularly limited, and can be freely selected as appropriate. For example, a blood sample can be used. Note that in the present technology, the “blood sample” only needs to be a sample containing red blood cells and liquid components such as plasma, and is not limited to blood itself. More specifically, examples of the “blood sample” include a blood sample containing blood components such as whole blood, plasma, and dilutions thereof, and/or an agent additive. Examples of the agent include an anticoagulant and an agent against the anticoagulant. More specifically, examples of the agent include a calcium aqueous solution, various blood clotting factors, various coagulants, a heparin neutralizing agent, a fibrinolytic system inhibitor, a platelet inhibitor, and a platelet activating agent.

The electrical characteristics measurement apparatus 100 according to the present technology is capable of favorably measuring the electrical characteristics of, particularly, the biological sample S in a liquid state or a gel state.

For example, in the case where the biological sample S is a blood sample, the value measured as the electrical characteristics by the measurement unit 1 can be appropriately selected depending on the purpose of analysis of the blood sample, such as analysis of the blood clotting function. More specifically, for example, the value may be, for example, a value of impedance, a value of dielectric constant, or the like. In the present technology, among these, the electrical characteristics may be, particularly, a dielectric constant at a specific frequency.

Further, the configuration of the measurement unit 1 can be freely designed as appropriate as long as the measurement unit 1 is configured to be capable of measuring the electrical characteristics of the biological sample S to be measured. For example, in the case of measuring impedance and a dielectric constant as the electrical characteristics, an impedance analyzer, a network analyzer, or the like can be adopted as the measurement unit 1.

More specifically, for example, the measurement unit 1 may be configured to measure the impedance of the biological sample S acquired by applying alternating voltage to the biological sample S by an application unit 120 to be described later, and measure the impedance of the biological sample S between electrodes 121a and 121b with the start time of the time point when an instruction to start measurement is received or the time point when the power of the apparatus 100 is turned on. Then, a dielectric constant and the like can be derived from the measured impedance. In order to derive the dielectric constant, a well-known function or relational formula showing the relationship between the impedance and the dielectric constant can be used.

Further, the measurement unit 1 is also capable of performing multiple measurement. Examples of the method of performing the multiple measurement include a method of simultaneously performing the multiple measurement with a plurality of provided measurement units 1, a method of performing the multiple measurement by causing one measurement unit 1 to perform scanning, a method of performing the multiple measurement by causing a biological sample holding unit 110 to be described later to move, and a method of selecting, by switching, one or more measurement units 1 that actually perform measurement from the plurality of provided measurement units 1.

(a) Biological Sample Holding Unit 110

The measurement unit 1 may include the biological sample holding unit 110. The biological sample holding unit 110 is a section that holds the biological sample S to be measured.

In the electrical characteristics measurement apparatus 100 according to the present technology, the number of biological sample holding units 110 is not particularly limited, and one or more biological sample holding units 110 can be freely arranged depending on the amount or type of the biological sample S to be measured, the measurement purpose, and the like.

In the electrical characteristics measurement apparatus 100 according to the present technology, the electrical characteristics are measured while the biological sample holding unit 110 holds the biological sample S. For that reason, the biological sample holding unit 110 is favorably configured to be sealable while holding the biological sample S. However, the biological sample holding unit 110 does not necessarily need to be hermetic as long as it is capable of staying for the time required for measuring the electrical characteristics of the biological sample S and there is no influence on the measurement.

A specific method of introducing the biological sample S into the biological sample holding unit 110 and a specific method of sealing the biological sample holding unit 110 are not particularly limited, and the biological sample S can be introduced by a free method depending on the form of the biological sample holding unit 110. Examples of such a method include a method of sealing the biological sample holding unit 110 by providing a lid portion in the biological sample holding unit 110, introducing the biological sample S by using a pipette or the like, and then closing the lid portion, and a method of sealing the biological sample holding unit 110 by introducing an injection needle from the outer surface of the biological sample holding unit 110, injecting the liquid biological sample S, and then blocking, with grease or the like, the part through which the injection needle has penetrated.

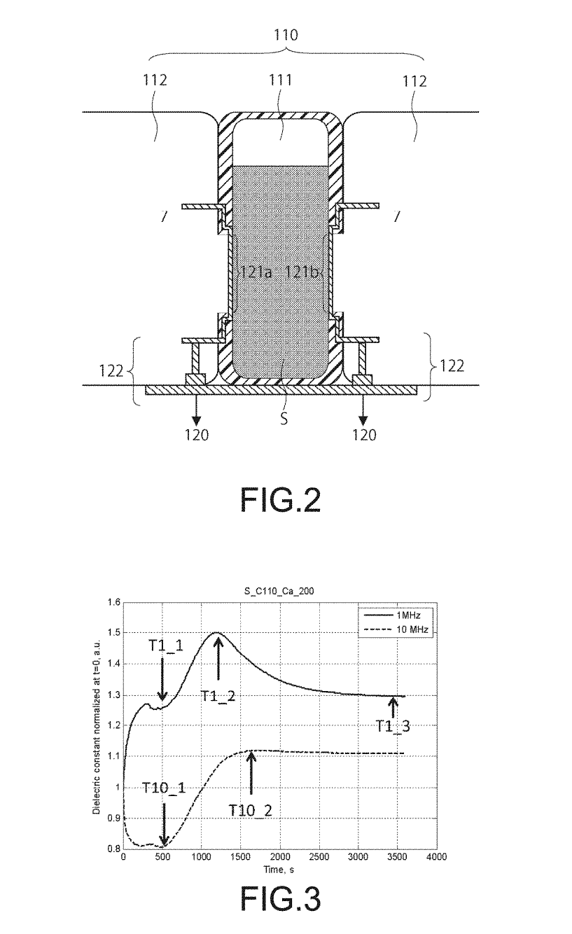

The form of the biological sample holding unit 110 is not particularly limited as long as the biological sample S to be measured can be held in the apparatus, and can be designed in a free form. For example, one or more cells provided on a substrate can be made to function as the biological sample holding unit 110, or one or more containers can be made to function as the biological sample holding unit 11. Hereinafter, one form of the biological sample holding unit 110 will be described with reference to FIG. 2.

FIG. 2 is a schematic cross-sectional view schematically showing a form of the biological sample holding unit 110. The biological sample holding unit 110 shown in FIG. 2 includes a container 111 and a container holding unit 112.

Note that in the electrical characteristics measurement apparatus 100 according to the present technology, by designing a container holding unit 110 so that a well-known cartridge type container for measurement can be used as the container 111, it is possible to cause only the container holding unit 112 to function as the biological sample holding unit 110. That is, in the present technology, the biological sample holding unit 110 includes only the container 111, includes the container 111 and the container holding unit 112, or includes only the container holding unit 112.

(a-1) Container 111

In the case of using the container 111 as the biological sample holding unit 110, the specific form thereof is not particularly limited and can be freely designed as appropriate depending on the state or type of the biological sample S, and the like as long as the biological sample S to be measured can be held. Examples of the specific form include a cylindrical body, a polygonal cylinder with a polygonal (triangular, square or more) cross section, a cone, a polygonal pyramid with a polygonal (triangular, square or more) cross section, and a combination of one or more kinds thereof.

Further, also the material forming the container 111 is not particularly limited, and can be freely selected within a range that does not affect the state or type of the biological sample S to be measured, the measurement purpose, and the like. In particular, in the present technology, it is favorable to use resin to form the container 111 from the viewpoint of easy of processing, and the like. In the present technology, also the type of resin that can be used is not particularly limited, and one or more kinds of resin applicable for holding the biological sample S can be freely selected as appropriate and used. Examples of the resin include hydrophobic and insulating polymers such as polypropylene, polymethyl methacrylate, polystyrene, acrylic, polysulfone, and polytetrafluoroethylene, copolymers, and blended polymers.

In the present technology, among these, it is particularly favorable to form the biological sample holding unit 110 with one or more kinds of resin selected from polypropylene, polystyrene, acrylic, and polysulfone. Since these kinds of resin have low coagulation activity against blood, they can be favorably used for measuring the blood sample.

(a-2) Container Holding Unit 112

In the case of using the container holding unit 112 as the biological sample holding unit 110, the specific form thereof is not particularly limited and can be freely designed as long as it is capable of holding the container 111 containing the biological sample S to be measured.

Further, also the material forming the container holding unit 112 is not particularly limited and can be freely selected depending on the form of the container 111 held by the container holding unit 112, and the like.

(b) Application Unit 120

The measurement unit 1 may include the application unit 120. The application unit 120 is a section that applies alternating voltage to a pair of electrodes 121a and 121b in contact with the biological sample S held by the biological sample holding unit 110. More specifically, for example, the application unit 120 applies voltage to the pair of electrodes 121a and 121b at the start time of the time point when an instruction to start measurement is received or the time point when the power of the apparatus 10 is turned on. More specifically, the application unit 120 applies alternating voltage of a set frequency or a frequency controlled by the measurement condition control unit 6 to be described later to the electrodes 121a and 121b for each set measurement interval or each measurement interval controlled by the measurement condition control unit 6 to be described later.

(b-1) Electrodes 121a and 121b

The electrodes 121a and 121b are brought into contact with the biological sample S at the time of measurement, and used for applying necessary voltage to the biological sample S. In the present technology, the number of electrodes 121a and 121b is not particularly limited as long as they are capable of measuring the impedance of the biological sample S, and more than one pair of electrodes can be freely arranged.

Further, also the arrangement, form, and the like of the electrodes 121a and 121b are not particularly limited, and the electrodes 121a and 121b can be freely designed as appropriate depending on the form of the biological sample holding unit 110, and the like as long as they are capable of applying necessary voltage to the biological sample S. For example, as in the biological sample holding unit 110 shown in FIG. 2, the electrodes 121a and 121b may be integrated with the biological sample holding unit 110 (container 111). Alternatively, although not shown, by providing the electrodes 121a and 121b on the lid portion of the container 111 and sealing the electrodes 121a and 121b with the lid portion, the electrodes 121a and 121b may be brought into contact with the biological sample S contained in the container 111. Alternatively, by inserting the pair of electrodes 121a and 121b from the outside of the container 111 into the container 111 at the time of measurement, the electrodes 121a and 121b may be brought into contact with the biological sample S.

Also the material forming the electrodes 121a and 121b is not particularly limited, and one or more well-known electrically conductive materials can be freely selected as appropriate within a range that does not affect the state or type of the biological sample S to be measured, the measurement purpose, and the like, and used. Examples of the material include titanium, aluminum, stainless steel, platinum, gold, copper, and graphite.

In the present technology, among these, it is particularly favorable to form the electrodes 121a and 121b with an electrically conductive material containing titanium. Since titanium has low coagulation activity against blood, it can be favorably used for measuring the biological sample S.

(b-2) Connection Unit 122

A connection unit 122 is a section that electrically connects the application unit 120 and the electrodes 121a and 121b. The specific form of the connection unit 122 is not particularly limited, and can be designed in a free form as appropriate as long as it is capable of electrically connecting the application unit 120 and the electrodes 121a and 121b.

(2) Analysis Unit 2

The analysis unit 2 is a section that reviews data related to the temporal change in electrical characteristics in real time during the measurement in the measurement unit 1 and analyzes a change in state of the biological sample S. The analysis unit 2 detects a predetermined feature point from the data related to the temporal change in electrical characteristics and uses data related to the temporal change in a period before and/or after the predetermined feature point. Specifically, for example, processing as shown in analysis examples 1 to 3 to be described later is performed. Accordingly, it is possible to cover an algorithm keeping real-time properties and thus return an analysis result to the user during the measurement at the time as close as possible to the real time. Further, the risk of erroneous determination resulting from the change in state of the biological sample S or the like can be reduced.

In the case where the biological sample S is a blood sample, in the present technology, specific examples of the state of an analyzable blood sample are not particularly limited as long as it has a phenomenon that temporal changes of impedance, a dielectric constant, and the like are observable from the state change, and various state changes can be detected and analyzed. Examples of the state change include the sedimentation of red blood cells, clotting (coagulation) of blood, fibrin formation, fibrin clot formation, blood clot formation, platelet agglutination, rouleaux formation of red blood cells, blood agglutination, clot retraction, hemolysis such as fibrinolysis, and fibrinolysiss.

Hereinafter, the analysis performed by the analysis unit 2 will be described using a specific example.

### Analysis Example 1 Performed by Analysis Unit 2

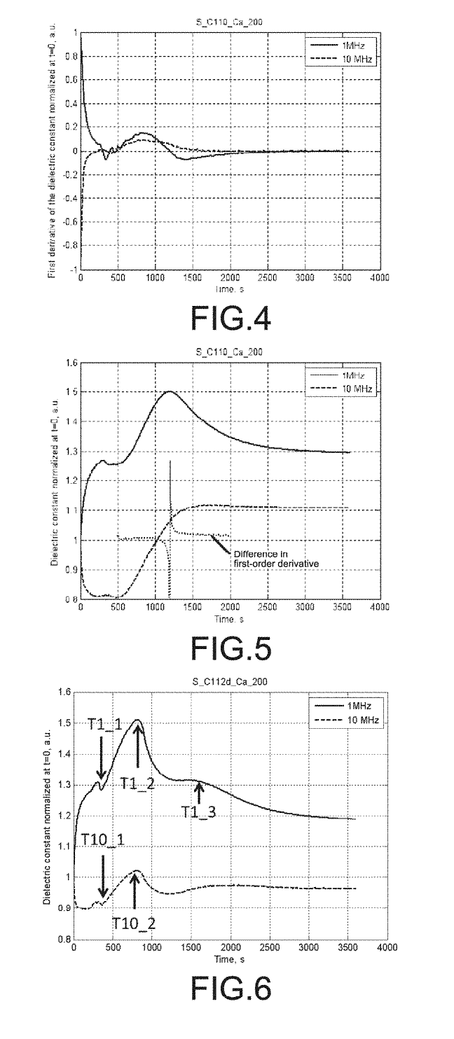

FIG. 3 is a drawing substitute graph showing data related to the temporal change (blood clotting curve) at the dielectric constants of 1 MHz and 10 MHz in the case of a typical blood clotting process. FIG. 4 is a drawing substitute graph showing a first-order derivative of 1 MHz and a first-order derivative of 10 MHz in the case of a typical blood clotting process. Further, FIG. 5 is a drawing substitute graph showing data related to the temporal change and a difference in first-order derivative in the case of a typical blood clotting process. Note that points indicated by the arrows in FIG. 3 represent predetermined feature points (basics of analysis output information).

In FIG. 3, information obtained before T1_1 is related to the rouleaux of red blood cells. “T1_1” represents the start point of increase in dielectric constant associated with clotting in 1 MHz, “T10_1” represents the start point of increase in dielectric constant associated with clotting in 10 MHz, “T1_2” represents the start point of decrease in dielectric constant associated with clotting in 1 MHz, “T10_2” represents the end point of increase in dielectric constant associated with clotting in 1 MHz, “T1_3” represents the end point of decrease in dielectric constant associated with clotting in 1 MHz, and they are specific examples of the “predetermined feature points” in the present technology. Note that in this specification, the definition of them (T1_1, T10_1, T1_2, T10_2, T1_3) is the same in the analysis examples 2 and 3 and measurement method examples 1 to 4 that will be described later.

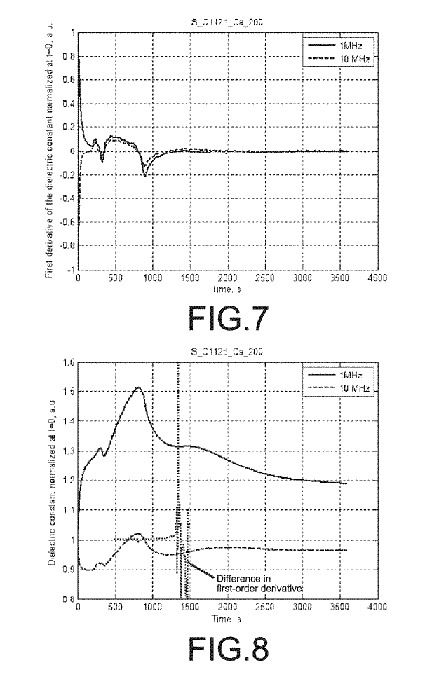

Meanwhile, FIG. 6 is a drawing substitute graph showing data related to the temporal change at the dielectric constants of 1 MHz and 10 MHz in the case where an erythrocyte sedimentation rate (sedimentation) is abnormal. FIG. 7 is a drawing substitute graph showing a first-order derivative of 1 MHz and a first-order derivative of 10 MHz in the case where the erythrocyte sedimentation rate (sedimentation) is abnormal. Further, FIG. 8 is a drawing substitute graph showing data related to the temporal change and a difference in first-order derivative in the case where the erythrocyte sedimentation rate (sedimentation) is abnormal. Note that points indicated by the arrows in in FIG. 6 represent predetermined feature points (basics of analysis output information).

In this analysis example 1, the analysis is performed on the basis of the following definition in FIGS. 3 and 6. More specifically, time points at which the first derivatives of 1 MHz and 10 MHz become larger than set threshold values (Thresh_1+>0, Thresh_10+>0), respectively, are analyzed at T1_1 and T10_1, a time point at which the first derivative of 1 MHz becomes smaller than a set threshold value (Thresh_1−<0) is analyzed at T1_2, and a time point at which the first derivative of 1 MHz becomes larger than a set threshold value (Thresh_1−<0) is analyzed at T1_3. Here, it can be said that the case of FIG. 3 as described above reflects a typical blood clotting process without particularly causing any problem. However, the case shown as in FIG. 6 could detect T1_2 while failing to reflect the blood clotting process at that time point and reflecting a process of the sedimentation of red blood cells.

Further, as shown in FIG. 4, in the case of the typical blood clotting process, a phenomenon that the dielectric constants uniformly vary irrespective of the frequency is not observed. Therefore, as shown in FIG. 4, the first-order derivatives of 1 MHz and 10 MHz have a large difference therebetween in the vicinity of T1_2. Meanwhile, as shown in FIG. 7, in the case where the sedimentation is abnormal, a phenomenon that the dielectric constants uniformly vary in a wide frequency (at least 500 kHz to 10 MHz) is observed. Therefore, as shown in FIG. 7, the variations of the first-order derivatives of 1 MHz and 10 MHz almost coincide with each other in the vicinity of T1_2.

Moreover, in the case of the typical clotting process as shown in FIG. 5, a large difference in first-order derivatives of 1 MHz and 10 MHz is seen in a period around T1_2, whereas in the case where the sedimentation is abnormal as shown in FIG. 8, a small difference in first-order derivatives of 1 MHz and 10 MHz is seen in the period around T1_2 (in a time zone at around 800 seconds).

From the description above, for example, by using the data related to the temporal change at a plurality of frequencies of 1 MHz and 10 MHz, analyzing in the analysis unit 2 a time point at which a value at the predetermined feature point exceeds a predetermined threshold value (e.g., a time point at which the first derivative at T1_2 becomes smaller than a set threshold value (Thresh_1−<0) in the analysis example 1), and comparing the variations in electrical characteristics at the time point between the pieces of data related to the temporal change, it is possible to determine that the sedimentation is abnormal in the blood clotting process.

Note that, in the present technology, the method of comparing the variations in electrical characteristics is not particularly limited. For example, the comparison may be performed by using the difference in first-order derivative as described above or can be performed by verifying the ratio of the first-order derivatives, for example.

### Analysis Example 2 Performed by Analysis Unit 2

In this analysis example 2, in addition to the calculation of the first-order derivative by the same flow as that of the above-mentioned analysis example 1, a correlation coefficient between the frequencies of 1 MHz and 10 MHz is obtained to perform analysis. Specifically, for example, T1_2 is detected, the measurement is performed for one minute, and a correlation coefficient between 1 MHz and 10 MHz is calculated on the basis of the measurement from two minutes before the T1_2 to one minute after the T1_2. In the case where the correlation coefficient is larger than a set threshold value (e.g., 0.95), it is determined that the sedimentation is abnormal, and the clotting analysis is terminated.

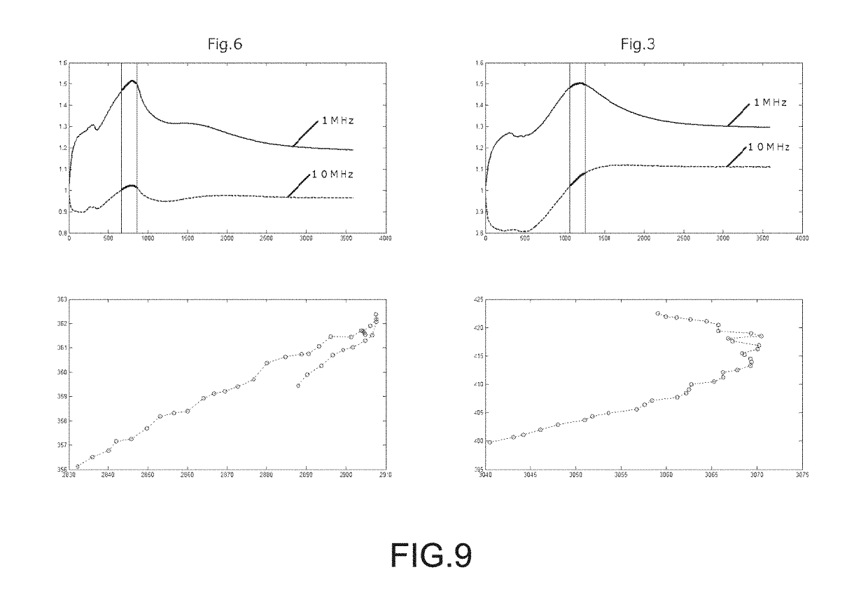

FIG. 9 is a drawing substitute graph showing calculation results in the case where tbuffer=12 in the data related to the temporal change shown in FIGS. 3 and 6. In each of the upper graphs of FIG. 9, a gap between the two thick, vertical lines represents the period used for the analysis. In FIG. 9, the lower-left graph shows results obtained in the case where tbuffer=12 in the data (on the upper left) of FIG. 6, the vertical axis represents a change in dielectric constant of 10 MHz in the period used for the analysis, and the horizontal axis represents a change in dielectric constant of 1 MHz in the period used for the analysis. In the lower-left graph of FIG. 9, the correlation coefficient is 0.98, and a correlation between the case of the frequency of 1 MHz and the case of the frequency of 10 MHz is seen. Meanwhile, in FIG. 9, the lower-right graph shows results obtained in the case where tbuffer=12 in the data (on the upper right) of FIG. 3, the vertical axis represents a change in dielectric constant of 10 MHz in the period used for the analysis, and the horizontal axis represents a change in dielectric constant of 1 MHz in the period used for the analysis. In this case, a correlation is not seen therebetween.

From the description above, for example, by calculating the correlation coefficient between the data related to the temporal change at plurality of frequencies of 1 MHz and 10 MHz, analyzing whether the correlation coefficient exceeds a predetermined threshold value or not (e.g., whether the correlation coefficient is larger than the set threshold value (0.95) or not in the analysis example 2), and comparing the variations in electrical characteristics at the above-mentioned time point between the pieces of data related to the temporal change, it is possible to determine that the sedimentation is abnormal in the blood clotting process.

### Analysis Example 3 Performed by Analysis Unit 2

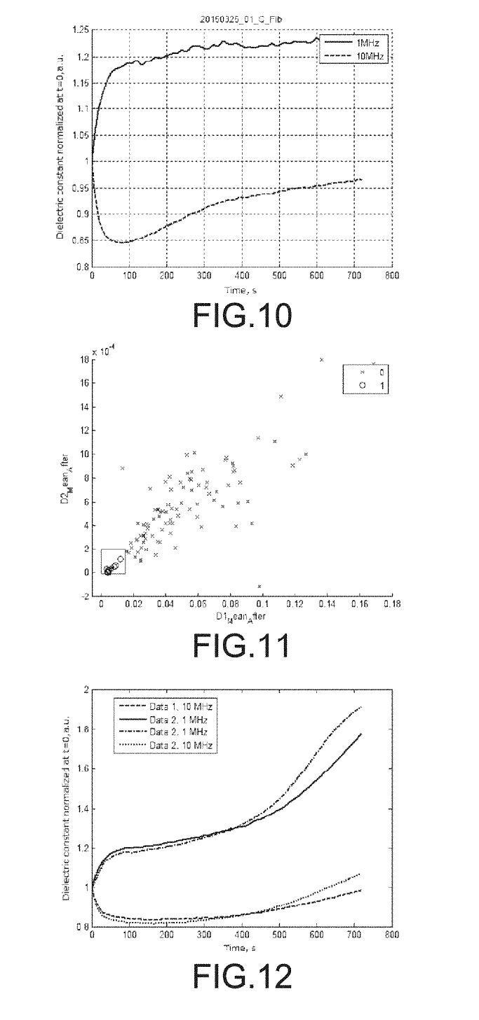

In the data related to the temporal change of a blood sample in which the sedimentation of red blood cells progresses very slowly and the blood is basically not clotted (i.e., data showing that blood is not clotted and showing low sedimentation), as shown in FIG. 10, there is a possibility that an erroneous detection of T10_1 occurs. In the data shown in FIG. 10, the decrease in dielectric constant of 1 MHz as observed in a normal specimen (a blood sample of a non-handicapped person) is not found, but the increase in dielectric constant of 10 MHz is observed. In such data, T10_1 is calculated and output, but this increase in dielectric constant of 10 MHz is not due to blood clotting, but due to the sedimentation of red blood cells. This is because, if the increase is due to blood clotting, the decrease in dielectric constant of 1 MHz should also be observed later. However, since it is necessary to perform such data related to the temporal change in real time, the case as described above will be examined in this analysis example 3.

As shown in the above-mentioned analysis example 2, T10_1 is determined, and analysis is performed with a first-order derivative of T10_1 and a second-order derivative of T10_1 after the elapse of a predetermined time. FIG. 11 is a drawing substitute graph showing analysis results of a first-order derivative at two minutes after T10_1 and a second-order derivative at two minutes after T10_1. In FIG. 11, the vertical axis represents a mean of the second-order derivatives of 10 MHz at two minutes after T10_1, and the horizontal axis represents a mean of the first-order derivatives of 10 MHz at two minutes after T10_1. In FIG. 11, data points of “x” and “∘” are obtained by visually determining whether a value at the predetermined feature point and/or the state of the biological sample at the predetermined feature point are/is matched with a predetermined criterion or not. In the case where it is not matched with the predetermined criterion, such a case is determined as “x” (determined as normal blood clotting data), and in the case where it is matched with the predetermined criterion, such a case is determined as “∘” (determined as data showing that blood is not clotted and showing low sedimentation). In FIG. 11, the data enclosed by a square includes almost only the data points of “∘”, that is, it can be said that the analysis can be performed with those two parameters.

Note that, in the present technology, examples of the above-mentioned technique of determining “whether a value at the predetermined feature point and/or the state of the biological sample at the predetermined feature point are/is matched with a predetermined criterion or not” include a technique of using data related to the temporal change obtained when the analysis is performed for a predetermined time (e.g., one hour) and determining that such a specimen that does not show the decrease of 1 MHz in the data after the elapse of a predetermined time (e.g., after the elapse of one hour) and shows the sedimentation of red blood cells when the measured specimen is visually observed is matched with a determination criterion.

In the present technology, the analysis unit 2 may perform analysis by using both of the two parameters (i.e., for example, the first-order derivative at two minutes after T10_1 and the second-order derivative at two minutes after T10_1 in the analysis example 3), or may perform analysis by using any of the parameters.

Note that the data enclosed by a square includes two data points of “x”, which are erroneously detected. However, as shown in FIG. 12, even if the data related to the temporal change exhibits a curved shape as in blood clotting in the graph as a whole, determination for blood clotting cannot be yet made at the time point of the determination at T10_1 because the blood clotting is very slow. In such a case, it is conceivable that after erroneous determination is made at T10_1, the analysis is subsequently performed, and correct determination at T10_1 can be made. To cope with this, for example, a method of setting the flow of the analysis and other methods can be adopted as shown in the measurement method example 3 to be described later, such that the analysis is not terminated after the determination as in the case of the above-mentioned analysis examples 1 and 2 where abnormal sedimentation is detected.

From the description above, it is possible to determine that the blood is clotted in the blood clotting process by analyzing whether the value at the predetermined feature point and/or the state of the biological sample at the predetermined feature point are/is matched with a predetermined criterion or not.

With the summary of the above-mentioned analysis examples 1 to 3, for example, the analysis unit 2 can perform determination on the basis of the following criteria.

TABLE 1

| | Blood clotting rate is relatively |
|---|---|
| Blood clotting rate is relatively slow | fast and sedimentation |
| and sedimentation is relatively slow | is relatively slow |
| Analysis example 3: | |
| → Blood is not clotted | → Normal measurement |
| | Blood clotting rate is relatively |
| Blood clotting rate is relatively slow | fast and sedimentation |
| and sedimentation is relatively fast | is relatively fast |
| Analysis examples 1 and 2: | |
| → Sedimentation is abnormal | → Normal measurement |

(3) Notification Unit 3

The notification unit 3 is a section that notifies the analysis result by the analysis unit 2 at a specific time point. For example, in the case where the biological sample S is a blood sample, the notification unit 3 generates a notification signal only in the case where the analysis result shown in Table 1 or the like is obtained during measurement, and notifies a user of the result in real time. With this configuration, since the user is notified of the analysis result only at the specific time point when the analysis result has been determined, the user does not need to see a result erroneously determined, and the usability is improved.

In the present technology, the configuration of the notification unit 3 is not particularly limited and can be freely designed as long as it is configured to notify the user of the analysis result of the analysis unit 2 at a particular time point.

Also the method of notifying the user is not particularly limited. For example, the user may receive the notification via the display unit 4 to be described later, a display, a printer, a speaker, lighting, or the like. Further, for example, a device having a communication function of transmitting an e-mail or the like for notifying that a notification signal has been generated to a mobile device such as a cellular phone and a smartphone may be used together with the notification unit 3.

(4) Display Unit 4

The electrical characteristics measurement apparatus 100 may further include the display unit 4. The display unit 4 is a section that displays the data related to the temporal change in electrical characteristics measured by the measurement unit 1, the analysis result by the analysis unit 2, the notification result from the notification unit 3, and the like. The configuration of the display unit 4 is not particularly limited. For example, as the display unit 4, a display, a printer, or the like can be adopted. Further, in the present technology, the display unit 4 is not necessarily needed to be provided, and an external display apparatus may be connected.

(5) Storage Unit 5

The electrical characteristics measurement apparatus 100 may further include the storage unit 5. The storage unit 5 is a section that stores the data related to the temporal change in electrical characteristics measured by the measurement unit 1, the analysis result by the analysis unit 2, the notification result from the notification unit 3, and the like. The configuration of the storage unit 5 is not particularly limited. For example, as the storage unit 5, a hard disk drive, a flash memory, an SSD (Solid State Drive), or the like can be adopted. Further, in the present technology, the storage unit 5 is not necessarily needed to be provided, and an external storage apparatus may be connected.

Further, in the present technology, an operation program and the like of the electrical characteristics measurement apparatus 100 may be stored in the storage unit 5. For example, the storage unit 5 may have a function of outputting specific parameters (e.g., blood clotting parameter) as shown in the measurement method examples 1 to 4 to be described later.

(6) Measurement Condition Control Unit 6

The electrical characteristics measurement apparatus 100 may further include the measurement condition control unit 6. The measurement condition control unit 6 is a section that controls the measurement time and/or the measurement frequency, and the like in the measurement unit 1.

As a specific method of controlling the measurement time, the measurement interval can be controlled depending on the amount of data necessary for the target analysis, and the like, or the timing of finishing the measurement can be controlled in the case where, for example, the measurement value has been substantially leveled off.

Further, it is also possible to control the measurement frequency depending on the type of the biological sample S to be measured, the measurement value necessary for the target analysis, and the like. Examples of the method of controlling the measurement frequency include a method of changing the frequency of alternating voltage to be applied between the electrodes 121a and 121b, and a method of superimposing a plurality of frequencies to measure the impedance at the plurality of frequencies. Examples of the specific method include a method of arranging a plurality of single-frequency analyzers side by side, a method of sweeping a frequency, a method of superimposing frequencies and extracting information of each frequency with a filter, and a method of performing measurement by using the response to impulse.

(7) Temperature Control Unit 7

The electrical characteristics measurement apparatus 100 may further include the temperature control unit 7. The temperature control unit 7 is a section that controls the temperature in the biological sample holding unit 110. In the electrical characteristics measurement apparatus 100 according to the present technology, this temperature control unit 7 is not an essential section. However, in order to keep the biological sample S to be measured in an optimal state for measurement, it is favorable to provide the temperature control unit 7.

Further, in the case of providing the sample standby unit 14 as will be described later, the temperature control unit 7 may control the temperature in the sample standby unit 14. Further, in the case where an agent is put in the biological sample S at the time of or before the measurement, the temperature control unit 6 may be provided to control the temperature of the agent. In this case, the temperature control units 6 may be provided for the temperature control in the biological sample holding unit 110, the temperature control in the sample standby unit 12, and the temperature control of the agent. Alternatively, one temperature control unit 6 may perform all the temperature control.

The specific method of controlling the temperature is not particularly limited. However, for example, by providing the container holding unit 112 with a temperature adjustment function, the container holding unit 112 can be made function as the temperature control unit 7.

(8) Biological Sample Supply Unit 8

The electrical characteristics measurement apparatus 100 may further include the biological sample supply unit 8. The biological sample supply unit 8 is a section that automatically supplies the biological sample holding unit 110 with the biological sample S. In the electrical characteristics measurement apparatus 100 according to the present technology, this biological sample supply unit 8 is not an essential section. However, by providing the biological sample supply unit 8, it is possible to automatically perform each step.

The specific method of supplying the biological sample S is not particularly limited. However, for example, in the case where the biological sample S is in a liquid state, it is possible to automatically supply the biological sample holding unit 110 with the biological sample S by using a pipetter and a tip attached to the end of the pipetter. In this case, in order to prevent measurement errors or the like from occurring, it is favorable that the tip is disposable. Further, it is also possible to automatically supply the biological sample S from the reservoir of the biological sample S to the biological sample holding unit 110 by using a pump or the like. Further, it is also possible to automatically supply the biological sample holding unit 110 with the biological sample S by using a permanent nozzle. In this case, in order to prevent measurement errors or the like from occurring, it is favorable to provide the nozzle with a cleaning function.

(9) Agent Supply Unit 9

The electrical characteristics measurement apparatus 100 may further include the agent supply unit 9. The agent supply unit 9 is a section that automatically supplies the biological sample holding unit 110 with one or more kinds of agents. In the electrical characteristics measurement apparatus 100 according to the present technology, this agent supply unit 9 is not an essential section. However, by providing the agent supply unit 9, it is possible to automatically perform each step.

The specific method of supplying the agent is not particularly limited, and a method similar to that of the biological sample supply unit 8 described above can be used. In particular, it is favorable to supply the agent by using a method capable of supplying a predetermined amount of agent without being in contact with the biological sample holding unit 110 (container 111). For example, in the case of a liquid agent, the agent can be discharged and supplied. More specifically, for example, it is possible to discharge and supply the liquid agent to the biological sample holding unit 110 (container 111) by introducing the liquid agent into a discharge pipe in advance and blowing, for a short time, pressurized air separately connected via a pipe line connected to the discharge pipe into the pipe line. At this time, by adjusting the air pressure and the valve opening/closing time, it is also possible to adjust the amount of liquid agent to be discharged.

Further, in addition to the blowing of air, it is also possible to discharge and supply the liquid agent to the biological sample holding unit 110 (container 111) by using vaporization of the liquid agent itself or air dissolved in it by heating. At this time, it is also possible to adjust the volume of generated bubbles and adjust the amount of liquid agent to be discharged by adjusting the voltage applied to a vaporizing chamber in which a heating element or the like is placed and the application time.

Further, it is possible to supply the biological sample holding unit 110 (container 111) with the liquid agent by driving a movable unit provided in the pipe line without using air but using a piezoelectric element (piezo element) or the like, and delivering the liquid agent in an amount determined by the volume of the movable unit. Further, for example, it is also possible to supply the agent by using a so-called inkjet method in which a liquid agent is made into fine droplets and sprayed directly onto the desired biological sample holding unit 110 (container 111).

The agent supply unit 9 may be provided with a stirring function, a temperature control function, an identification function (e.g., barcode reader) for identifying, for example, the type of the agent, and the like.

Note that in the case of using an agent, a predetermined agent in a solid state or in a liquid state as it is may be contained in the container 111 in advance. For example, in the case where the biological sample S containing the blood components is a target to be measured, an anticoagulant, a coagulation initiator, or the like can be contained in the container 111 in advance. By containing an agent in the container 111 in advance in such a manner, it is unnecessary to provide the agent supply unit 9 or a section that holds the agent, and it is possible to miniaturize the apparatus and reduce the cost. Further, since this reduces, for example, the trouble of the user to replace the agent and maintenance of the agent supply unit 9 or the agent holding unit is unnecessary, it is possible to improve the usability.

(10) Precision Management Unit 10

The electrical characteristics measurement apparatus 100 may further include the precision management unit 10. The precision management unit 10 is a section that manages precision of the measurement unit 1. In the electrical characteristics measurement apparatus 100 according to the present technology, this precision management unit 10 is not an essential section. However, by providing the precision management unit 10, it is possible to improve the precision of the measurement by the measurement unit 1.

The specific method of managing the precision of the measurement unit 1 is not particularly limited, and a well-known precision management method can be freely selected as appropriate and used. Examples of such a method include a method of managing the precision of the measurement unit 1 by performing calibration of the measurement unit 1, such as a method of performing calibration of the measurement unit 1 by placing a metal plate or the like for short-circuiting in the apparatus 100 and short-circuiting the electrode and the metal plate before starting measurement, a method of bringing a calibration jig or the like into contact with the electrode, and a method of performing calibration of the measurement unit 1 by placing a metal plate or the like in a container having the same form as that of the container 111 in which the biological sample S is to be put and short-circuiting the electrode and the metal plate before starting measurement.

Further, the present technology is not limited to the above-mentioned methods, and a free method, e.g., a method of managing the precision of the measurement unit 1 by checking the state of the measurement unit 1 before the actual measurement and calibrating the measurement unit 1 by performing the above-mentioned calibration or the like only when there is an abnormality, may be selected as appropriate and used.

(11) Drive Mechanism 11

The electrical characteristics measurement apparatus 100 may further include the drive mechanism 11. The drive mechanism 11 is a section to be used for moving the biological sample holding unit 110 in the measurement unit 1 depending on various purposes. For example, in the case where a blood sample is used as the biological sample S, by moving the biological sample holding unit 110 to the direction of changing the direction of gravity applied to the biological sample S held in the biological sample holding unit 110, it is possible to prevent the measurement value from being affected by sedimentation of the sedimentation component in the blood sample.

Further, for example, it is possible to drive the biological sample holding unit 110 so that the application unit 120 and the electrodes 121a and 121b can be disconnected from each other at the time of non-measurement and the application unit 120 and the electrodes 121a and 121b can be electrically connected to each other at the time of measurement as in the biological sample holding unit 110.

Further, for example, in the case of providing a plurality of biological sample holding units 110, by configuring the biological sample holding units 110 to be capable of moving, it is possible to perform measurement, biological sample supply, agent supply, and the like by moving the biological sample holding units 110 to necessary sections. That is, since it is unnecessary to move the measurement unit 1, the biological sample supply unit 8, the agent supply unit 9, and the like to the target biological sample holding unit 110, it is unnecessary to provide a drive unit or the like for moving the respective units and it is possible to miniaturize the apparatus and reduce the cost.

(12) Sample Standby Unit 12

The electrical characteristics measurement apparatus 100 may further include the sample standby unit 12. The sample standby unit 12 is a section in which the isolated biological sample S is caused to stand by before measurement. In the electrical characteristics measurement apparatus 100 according to the present technology, this sample standby unit 12 is not an essential section. However, by providing the sample standby unit 12, it is possible to smoothly measure the electrical characteristics.

The sample standby unit 12 may be provided with a stirring function, a temperature control function, a mechanism for moving to the biological sample holding unit 110, an identification function (e.g., barcode reader) for identifying, for example, the type of the biological sample S, an automatic opening function, and the like.

(13) Stirring Mechanism 13

The electrical characteristics measurement apparatus 100 may further include the stirring mechanism 13. The stirring mechanism 13 is a mechanism for stirring the biological sample S, and stirring the biological sample S and an agent. In the electrical characteristics measurement apparatus 100 according to the present technology, this stirring mechanism 13 is not an essential section. However, for example, in the case where the biological sample S contains a sedimentation component or the case where an agent is added to the biological sample S at the time of measurement, it is favorable to provide the stirring mechanism 13.

The specific stirring method by the stirring mechanism 13 is not particularly limited, and a well-known stirring method can be freely selected and used. Examples of such a method include stirring by pipetting, stirring using a stirring rod, a stirring bar, or the like, and stirring by reversing the container containing the biological sample S or the agent.

(14) Others

Note that functions performed by the respective units of the electrical characteristics measurement apparatus 100 according to the present technology may be stored as a program in a personal computer or a hardware resource including a control unit including a CPU and the like and a recording medium (non-volatile memory (USB memory or the like), HDD, CD, and the like), and implemented by the personal computer or the control unit.

2. Electrical Characteristics Measurement System 200

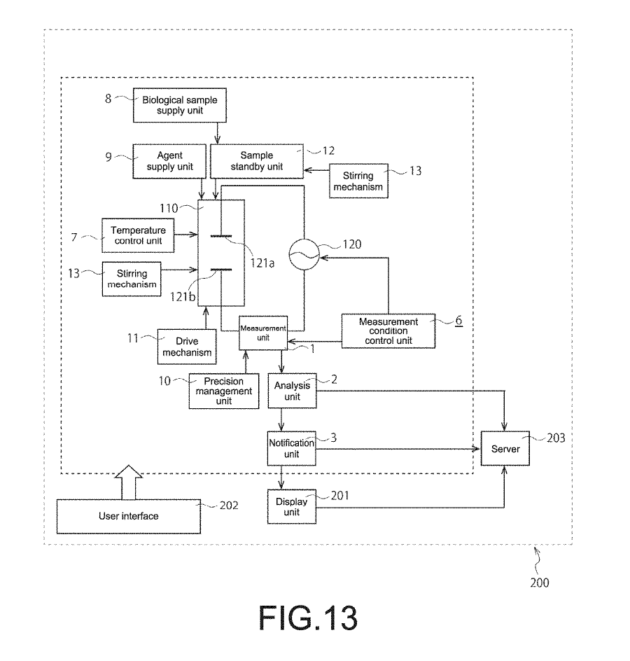

FIG. 13 is a schematic conceptual diagram schematically showing a concept of the electrical characteristics measurement system 200 according to the present technology. The electrical characteristics measurement system 200 according to the present technology roughly includes at least the measurement unit 1, the analysis unit 2, and the notification unit 3. Further, the electrical characteristics measurement system 200 may include, as necessary, the display unit 201, the user interface 202, the server 203, the measurement condition control unit 6, the temperature control unit 7, the biological sample supply unit 8, the agent supply unit 9, the precision management unit 10, the drive mechanism 11, the sample standby unit 12, the stirring mechanism 13, and the like. Hereinafter, the respective units will be described in detail. Note that since the measurement unit 1, the analysis unit 2, the notification unit 3, the measurement condition control unit 6, the temperature control unit 7, the biological sample supply unit 8, the agent supply unit 9, the precision management unit 10, the drive mechanism 11, the sample standby unit 12, and the stirring mechanism 13 are the same as those of the electrical characteristics measurement apparatus 100 described above, description thereof is omitted here.

(1) Display Unit 201

The display unit 201 is a section that displays the data related to the temporal change in electrical characteristics measured by the measurement unit 1, the analysis result by the analysis unit 2, the notification result by the notification unit 3, and the like. The configuration of the display unit 201 is not particularly limited. Note that FIGS. 3 to 12 described above show an example of the data displayed on the display unit 201.

Further, in the display unit 201, it is also possible to display the result of analyzing the physical properties or the state of the biological sample S, and the like by using the data related to the temporal change in electrical characteristics measured by the measurement unit 1.

(2) User Interface 202

The user interface 202 is a section for a user to operate. The user is capable of accessing the respective sections of the electrical characteristics measurement system 200 via the user interface 202.

(3) Server 203

The server 203 is a section that includes at least a storage unit that stores the data related to the temporal change acquired from the measurement unit 1 and/or the analysis result acquired from the analysis unit 2, and is connected to at least the measurement unit 1 and/or the analysis unit 2 via a network. It is also possible to improve the usability by providing the electrical characteristics measurement system 200 according to the present technology with this server 203.

Further, the server 203 is capable of managing various kinds of data uploaded from the respective sections of the electrical characteristics measurement system 200 and outputting the various kinds of data to the display unit 201 or the like according to an instruction from a user.

3. Electrical Characteristics Measurement Method

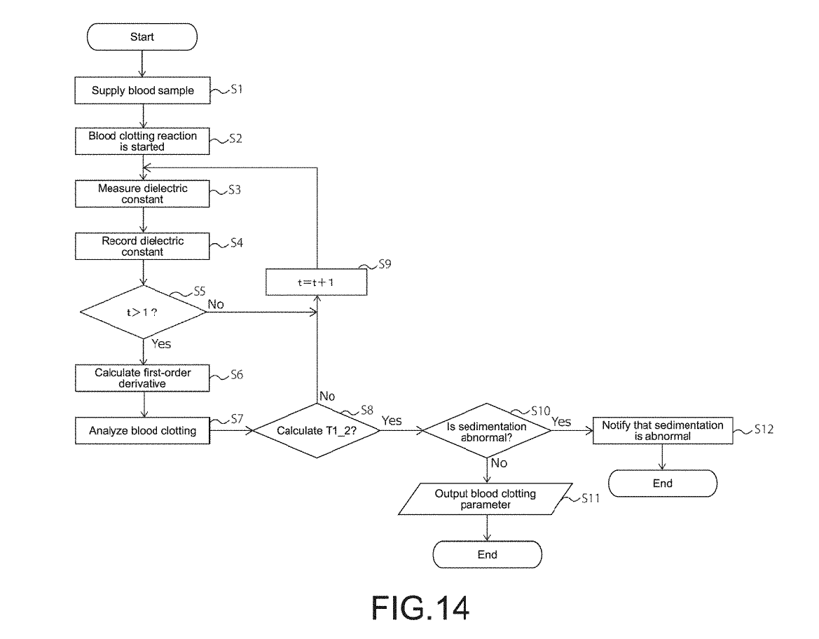

An electrical characteristics measurement method according to the present technology is a method including at least a measuring step, an analyzing step, and a notifying step. Since the specific method performed in the measuring step, the specific method performed in the analyzing step, and the specific method performed in the notifying step are respectively the same as the above-mentioned measurement method performed by the measurement unit 1 of the electrical characteristics measurement apparatus 100, the analysis method performed by the analysis unit 2 of the apparatus 100, and the notification method performed by the notification unit 3 of the apparatus 100, description thereof is omitted here. Hereinafter, examples of the measurement method using the electrical characteristics measurement method according to the present technology will be described with reference to FIGS. 14 to 17.

### Measurement Method Example 1

FIG. 14 is a flowchart showing an example of the electrical characteristics measurement method according to the present technology, and corresponds to the above-mentioned analysis example 1.

First, the biological sample supply unit 8 supplies a blood sample as the biological sample S (Step S1). Next, the agent supply unit 9 supplies an agent to start a blood clotting reaction, and blood clotting is started (Step S2). After that, the measurement unit 1 measures a dielectric constant with Index as a time (t) (Step S3), and the storage unit 5 records the dielectric constant (E (t)) (Step S4). Then, in the case where t>1 (Step S5), the analysis unit 2 calculates a first-order derivative (dE (t−1)) (Step S6) and analyzes blood clotting (Step S7). Meanwhile, in the case of not satisfying that t>1 (Step S5), the analysis unit 2 sets the expression t=t+1 (Step S9) and returns to Step S3.

After the analysis of the blood clotting by the analysis unit 2 (Step S7), in the case where the analysis unit 2 calculates T1_2 (Step S8), the analysis unit 2 determines whether sedimentation is abnormal or not by the method of comparing the difference between the first-order derivatives of 1 MHz and 10 MHz with the set threshold value as shown in the analysis example 1, for example (Step S10). Meanwhile, in the case where the analysis unit 2 does not calculate T1_2 (Step S8), the analysis unit 2 sets the expression t=t+1 (Step S9) and returns to Step S3.

After the analysis unit 2 determines that sedimentation is abnormal (Step S10), the notification unit 3 notifies the user of the fact that the sedimentation is abnormal (Step S12), and the processing is terminated. Meanwhile, in the case where the analysis unit 2 determines that the sedimentation is not abnormal (Step S10), the storage unit 5 outputs the blood clotting parameter (Step S11), and the processing is terminated.

### Measurement Method Example 2

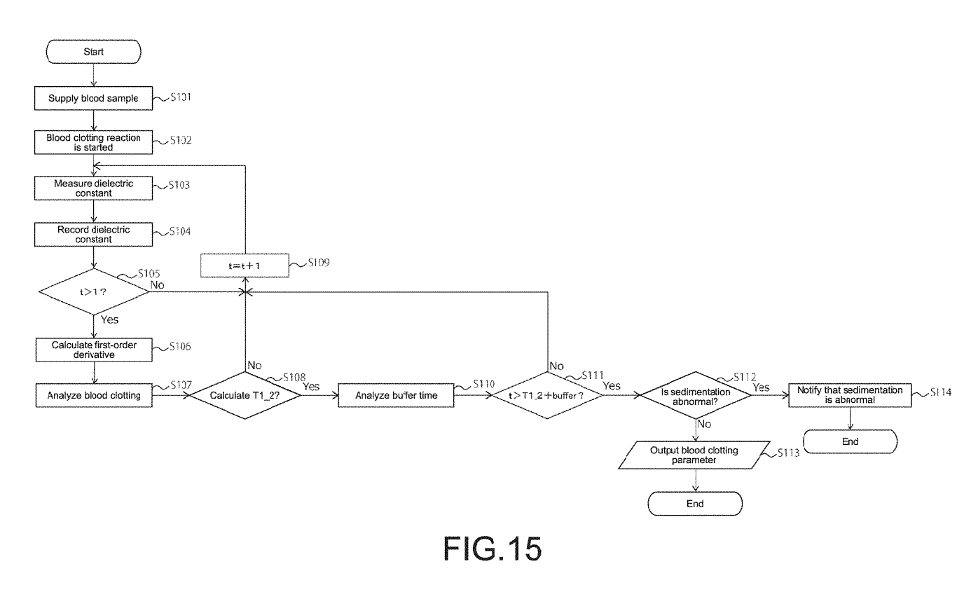

FIG. 15 is a flowchart showing an example of the electrical characteristics measurement method according to the present technology, which is different from that of FIG. 14 and corresponds to the above-mentioned analysis example 2.

First, the biological sample supply unit 8 supplies a blood sample as the biological sample S (Step S101). Next, the agent supply unit 9 supplies an agent to start a blood clotting reaction, and blood clotting is started (Step S102). After that, the measurement unit 1 measures a dielectric constant with Index as a time (t) (Step S103), and the storage unit 5 records the dielectric constant (E (t)) (Step S104). Then, in the case where t>1 (Step S105), the analysis unit 2 calculates a first-order derivative (dE (t−1)) (Step S106) and analyzes blood clotting (Step S107). Meanwhile, in the case of not satisfying that t>1 (Step S105), the analysis unit 2 sets the expression t=t+1 (Step S109) and returns to Step S103.

After the analysis of the blood clotting by the analysis unit 2 (Step S107), in the case where the analysis unit 2 calculates T1_2 (Step S108), the analysis unit 2 analyzes a buffer time as performed in the analysis example 2, for example (Step S110). After that, in the case where t>T1_2+buffer (Step S111), the analysis unit 2 determines whether sedimentation is abnormal or not (Step S112). Meanwhile, in the case where the analysis unit 2 does not calculate T1_2 (Step S108), the analysis unit 2 sets the expression t=t+1 (Step S109) and returns to Step S103. Further, also in the case of not satisfying that t>T1_2+buffer (Step S111), the analysis unit 2 sets the expression t=t+1 (Step S109) and returns to Step S103 in the similar manner.

After the analysis unit 2 determines that sedimentation is abnormal (Step S112), the notification unit 3 notifies the user of the fact that the sedimentation is abnormal (Step S114), and the processing is terminated. Meanwhile, in the case where the analysis unit 2 determines that the sedimentation is not abnormal (Step S112), the storage unit 5 outputs the blood clotting parameter (Step S113), and the processing is terminated.

### Measurement Method Example 3

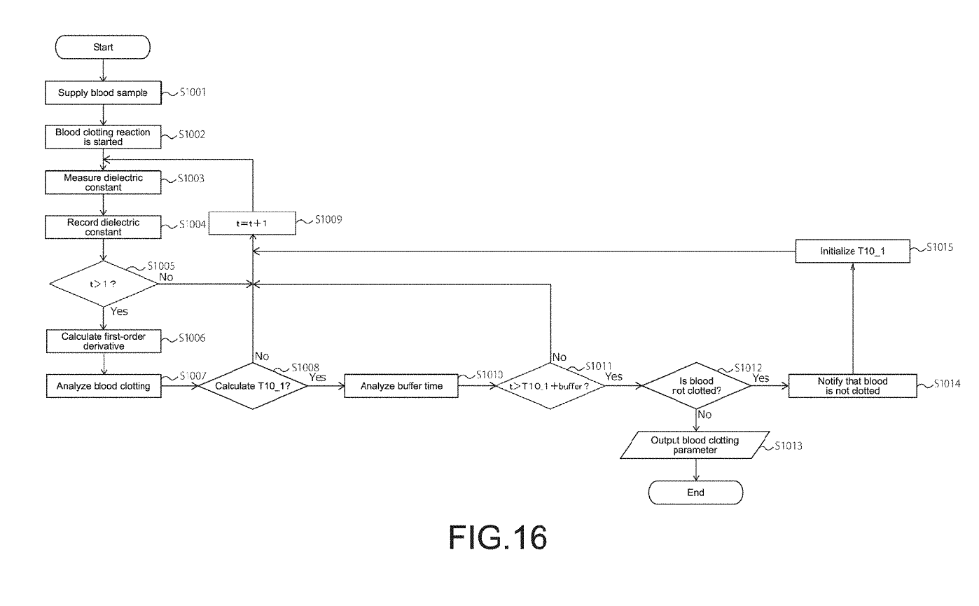

FIG. 16 is a flowchart showing an example of the electrical characteristics measurement method according to the present technology, which is different from those of FIGS. 14 and 15 and corresponds to the above-mentioned analysis example 3.

First, the biological sample supply unit 8 supplies a blood sample as the biological sample S (Step S1001). Next, the agent supply unit 9 supplies an agent to start a blood clotting reaction, and blood clotting is started (Step S1002). After that, the measurement unit 1 measures a dielectric constant with Index as a time (t) (Step S1003), and the storage unit 5 records the dielectric constant (E (t)) (Step S1004). Then, in the case where t>1 (Step S1005), the analysis unit 2 calculates a first-order derivative (dE (t−1)) (Step S1006) and analyzes blood clotting (Step S1007). Meanwhile, in the case of not satisfying that t>1 (Step S1005), the analysis unit 2 sets the expression t=t+1 (Step S1009) and returns to Step S1003.

After the analysis of the blood clotting by the analysis unit 2 (Step S1007), in the case where the analysis unit 2 calculates T10_1 (Step S1008), the analysis unit 2 analyzes a buffer time (Step S1010). After that, in the case where t>T10_1+buffer (Step S1011), the analysis unit 2 determines whether the blood is not clotted or not as performed in the analysis example 3 (Step S1012). Meanwhile, in the case where the analysis unit 2 does not calculate T10_1 (Step S1008), the analysis unit 2 sets the expression t=t+1 (Step S1009) and returns to Step S1003. Further, also in the case of not satisfying that t>T10_1+buffer (Step S1011), the analysis unit 2 sets the expression t=t+1 (Step S1009) and returns to Step S1003 in the similar manner.

After the analysis unit 2 determines that sedimentation is not clotted (Step S1012), the notification unit 3 notifies the user of the fact that the blood is not clotted (Step S1014). After the analysis unit 2 initializes T10_1 (Step S1015), the analysis unit 2 sets the expression t=t+1 (Step S1009) and returns to Step S1003. Meanwhile, in the case where the analysis unit 2 determines that the blood is not clotted (Step S1012), the storage unit 5 outputs the blood clotting parameter (Step S1013), and the processing is terminated.

### Measurement Method Example 4

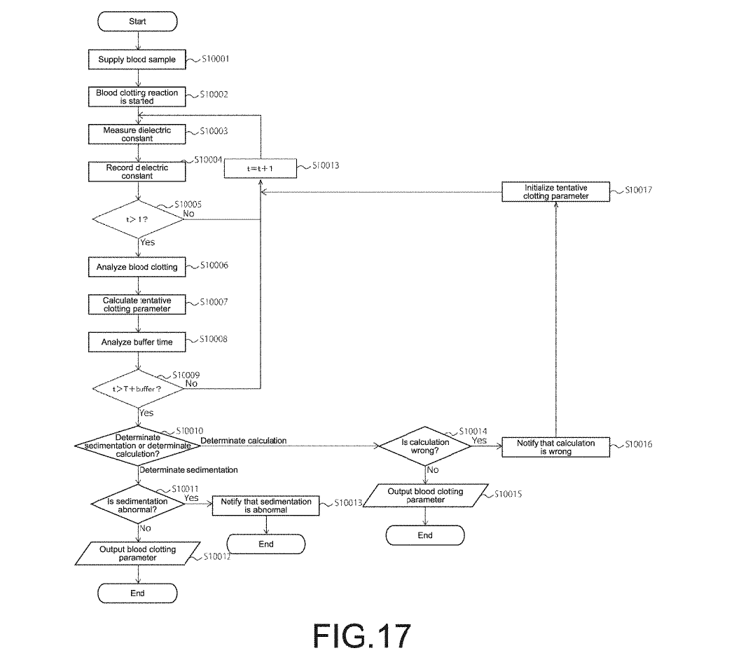

Note that the electrical characteristics measurement method according to the present technology can be performed by, as shown in FIG. 17, an embodiment including the above-mentioned flows performed in FIGS. 14 to 16.

First, the biological sample supply unit 8 supplies a blood sample as the biological sample S (Step S10001). Next, the agent supply unit 9 supplies an agent to start a blood clotting reaction, and blood clotting is started (Step S10002). After that, the measurement unit 1 measures a dielectric constant with Index as a time (t) (Step S10003), and the storage unit 5 records the dielectric constant (E (t)) (Step S10004). Then, in the case where t>1 (Step S10005), the analysis unit 2 analyzes blood clotting (Step S10006) and calculates a tentative clotting parameter (Step S10007). After that, the analysis unit 2 analyzes a buffer time (Step S10008).

After the analysis of the buffer time by the analysis unit 2 (Step S10008), the analysis unit 2 determines whether t>T (tentatively calculated time)+buffer (Step S10009). In the case where the analysis unit 2 determines that t>T+buffer (Step S10009), the analysis unit 2 determines whether to determine sedimentation or determine calculation (Step S10010). Meanwhile, in the case where the analysis unit 2 does not determine that t>T+buffer (Step S10009), the analysis unit 2 sets that t=t+1 (Step S10013) and returns to Step S10003.

In the case where the sedimentation is determined (Step S10011), the analysis unit 2 determines whether the sedimentation is abnormal or not (Step S10011). In the case where the analysis unit 2 determines that the sedimentation is abnormal (Step S10014), the notification unit 3 notifies the user of the fact that the sedimentation is abnormal (Step S10013), and the processing is terminated. Meanwhile, in the case where the analysis unit 2 determines that the sedimentation is not abnormal (Step S10011), the storage unit 5 outputs the blood clotting parameter (Step S10012), and the processing is terminated.

In the case where the calculation is determined (Step S10010), the analysis unit 2 determines whether the calculation is wrong or not (e.g., whether the blood is not clotted or not) (Step S10014). In the case where the analysis unit 2 determines that the calculation is wrong (Step S10014), the notification unit 3 notifies the user of the fact that the calculation is wrong (e.g., the blood is not clotted) (Step S10016). After the analysis unit 2 initializes the tentative clotting parameter (Step S10017), the analysis unit 2 sets the expression t=t+1 (Step S10013) and returns to Step S10003. Meanwhile, in the case where the analysis unit 2 determines that the calculation is not wrong (Step S10014), the storage unit 5 outputs the blood clotting parameter (Step S10015), and the processing is terminated.

Note that although the determination on sedimentation or the determination on calculation is performed in Step S10010 in the measurement method example 4 shown in FIG. 17, the present technology is not limited thereto. For example, the determination on calculation may be performed after the determination on sedimentation, or the determination on sedimentation may be performed after the determination on calculation.

The present technology can be have the following configurations.

(1)

An electrical characteristics measurement apparatus, including: at least

a measurement unit that measures electrical characteristics of a biological sample over time;

an analysis unit that reviews, in real time during the measurement, data related to a temporal change in the electrical characteristics and analyzes a change in state of the biological sample; and

a notification unit that issues notification of an analysis result in the analysis unit at a specific time point, in which

the analysis unit detects a predetermined feature point from the data related to the temporal change in the electrical characteristics to use data related to the temporal change in a period before and/or after the predetermined feature point.

(2)

The electrical characteristics measurement apparatus according to (1), in which

the analysis unit uses pieces of data related to the temporal change.

(3)

The electrical characteristics measurement apparatus according to (2), in which

the analysis unit detects a time point at which a value at the predetermined feature point exceeds a predetermined threshold value and compares variations in electrical characteristics at the time point between the pieces of data related to the temporal change.

(4)

The electrical characteristics measurement apparatus according to (2), in which

the analysis unit calculates a correlation coefficient between the pieces of data related to the temporal change and analyzes whether the correlation coefficient exceeds a predetermined threshold value or not.

(5)

The electrical characteristics measurement apparatus according to (1), in which

the analysis unit analyzes whether a value at the predetermined feature point is matched with a predetermined criterion or not.

(6)

The electrical characteristics measurement apparatus according to any one of (1) to (5), in which

the biological sample is a blood sample.

(7)

The electrical characteristics measurement apparatus according to any one of (1) to (6), in which

the electrical characteristics are a dielectric constant at a specific frequency.

(8)

An electrical characteristics measurement system, including: at least

a measurement unit that measures electrical characteristics of a biological sample over time;

an analysis unit that reviews, in real time during the measurement, data related to a temporal change in the electrical characteristics and analyzes a change in state of the biological sample; and

a notification unit that issues notification of an analysis result in the analysis unit at a specific time point, in which

the analysis unit detects a predetermined feature point from the data related to the temporal change in the electrical characteristics to use data related to the temporal change in a period before and/or after the predetermined feature point.

(9)

The electrical characteristics measurement system according to (8), further including

a server including at least a storage unit that stores the data related to the temporal change in the measurement unit and/or the analysis result in the analysis unit, the server being connected to the measurement unit and/or the analysis unit via a network.

(10)

An electrical characteristics measurement method, including: at least

a measuring step of measuring electrical characteristics of a biological sample over time;

an analyzing step of reviewing, in real time during the measurement, data related to a temporal change in the electrical characteristics and analyzing a change in state of the biological sample; and

a notifying step of issuing notification of an analysis result in the analysis unit at a specific time point, in which

the analyzing step including detecting a predetermined feature point from the data related to the temporal change in the electrical characteristics to use data related to the temporal change in a period before and/or after the predetermined feature point.

(11)

The electrical characteristics measurement method according to (10), in which

the analyzing step includes using pieces of data related to the temporal change.

(12)

The electrical characteristics measurement method according to (11), in which

the analyzing step includes detecting a time point at which a value at the predetermined feature point exceeds a predetermined threshold value and comparing variations in electrical characteristics at the time point between the pieces of data related to the temporal change.

(13)

The electrical characteristics measurement method according to (11), in which

the analyzing step includes calculating a correlation coefficient between the pieces of data related to the temporal change and analyzing whether the correlation coefficient exceeds a predetermined threshold value or not.

(14)

The electrical characteristics measurement method according to (10), in which

the analyzing step includes analyzing whether a value at the predetermined feature point is matched with a predetermined criterion or not.

(15)

A program that causes a computer to function as:

a measurement unit that measures electrical characteristics of a biological sample over time;

an analysis unit that reviews, in real time during the measurement, data related to a temporal change in the electrical characteristics and analyzes a change in state of the biological sample; and

a notification unit that issues notification of an analysis result in the analysis unit at a specific time point, in which

the analysis unit detects a predetermined feature point from the data related to the temporal change in the electrical characteristics to use data related to the temporal change in a period before and/or after the predetermined feature point.

### INDUSTRIAL APPLICABILITY

By using the present technology, it is possible to reduce the risk of erroneous determination while ensuring real-time properties in the measurement of electrical characteristics of a biological sample.

### REFERENCE SIGNS LIST

100: electrical characteristics measurement apparatus
1: measurement unit
110: biological sample holding unit
111: container
112: container holding unit
120: application unit
121a, 121b: electrode
122: connection unit
2: analysis unit
3: notification unit
4: display unit
5: storage unit
6: measurement condition control unit
7: temperature control unit
8: blood sample supply unit
9: agent supply unit
10: precision management unit
11: drive mechanism
12: sample standby unit
13: stirring mechanism
200: electrical characteristics measurement system
201: display unit
202: user interface
203: server
S: biological sample

1. An electrical characteristics measurement apparatus, comprising: at least
a measurement unit that measures electrical characteristics of a biological sample over time;
an analysis unit that reviews, in real time during the measurement, data related to a temporal change in the electrical characteristics and analyzes a change in state of the biological sample; and
a notification unit that issues notification of an analysis result in the analysis unit at a specific time point, wherein
the analysis unit detects a predetermined feature point from the data related to the temporal change in the electrical characteristics to use data related to the temporal change in a period before and/or after the predetermined feature point.

2. The electrical characteristics measurement apparatus according to claim 1, wherein
the analysis unit uses pieces of data related to the temporal change.

3. The electrical characteristics measurement apparatus according to claim 2, wherein
the analysis unit detects a time point at which a value at the predetermined feature point exceeds a predetermined threshold value and compares variations in electrical characteristics at the time point between the pieces of data related to the temporal change.

4. The electrical characteristics measurement apparatus according to claim 2, wherein
the analysis unit calculates a correlation coefficient between the pieces of data related to the temporal change and analyzes whether the correlation coefficient exceeds a predetermined threshold value or not.

5. The electrical characteristics measurement apparatus according to claim 1, wherein
the analysis unit analyzes whether a value at the predetermined feature point and/or the state of the biological sample at the predetermined feature point are/is matched with a predetermined criterion or not.

6. The electrical characteristics measurement apparatus according to claim 1, wherein
the biological sample is a blood sample.

7. The electrical characteristics measurement apparatus according to claim 1, wherein
the electrical characteristics are a dielectric constant at a specific frequency.

8. An electrical characteristics measurement system, comprising: at least
a measurement unit that measures electrical characteristics of a biological sample over time;
an analysis unit that reviews, in real time during the measurement, data related to a temporal change in the electrical characteristics and analyzes a change in state of the biological sample; and
a notification unit that issues notification of an analysis result in the analysis unit at a specific time point, wherein
the analysis unit detects a predetermined feature point from the data related to the temporal change in the electrical characteristics to use data related to the temporal change in a period before and/or after the predetermined feature point.

9. The electrical characteristics measurement system according to claim 8, further comprising
a server including at least a storage unit that stores the data related to the temporal change in the measurement unit and/or the analysis result in the analysis unit, the server being connected to the measurement unit and/or the analysis unit via a network.

10. An electrical characteristics measurement method, comprising: at least
a measuring step of measuring electrical characteristics of a biological sample over time;
an analyzing step of reviewing, in real time during the measurement, data related to a temporal change in the electrical characteristics and analyzing a change in state of the biological sample; and
a notifying step of issuing notification of an analysis result in the analysis unit at a specific time point, wherein
the analyzing step including detecting a predetermined feature point from the data related to the temporal change in the electrical characteristics to use data related to the temporal change in a period before and/or after the predetermined feature point.

11. The electrical characteristics measurement method according to claim 10, wherein
the analyzing step includes using pieces of data related to the temporal change.

12. The electrical characteristics measurement method according to claim 11, wherein
the analyzing step includes detecting a time point at which a value at the predetermined feature point exceeds a predetermined threshold value and comparing variations in electrical characteristics at the time point between the pieces of data related to the temporal change.

13. The electrical characteristics measurement method according to claim 11, wherein
the analyzing step includes calculating a correlation coefficient between the pieces of data related to the temporal change and analyzing whether the correlation coefficient exceeds a predetermined threshold value or not.

14. The electrical characteristics measurement method according to claim 10, wherein
the analyzing step includes analyzing whether a value at the predetermined feature point and/or the state of the biological sample at the predetermined feature point are/is matched with a predetermined criterion or not.

15. A program that causes a computer to function as:
a measurement unit that measures electrical characteristics of a biological sample over time;
an analysis unit that reviews, in real time during the measurement, data related to a temporal change in the electrical characteristics and analyzes a change in state of the biological sample; and
a notification unit that issues notification of an analysis result in the analysis unit at a specific time point, wherein
the analysis unit detects a predetermined feature point from the data related to the temporal change in the electrical characteristics to use data related to the temporal change in a period before and/or after the predetermined feature point.
